(19) United States
(12) Patent Application Publication
(10) Pub. No.: US 2024/0207245 A1
(43) Pub. Date: Jun. 27, 2024

(54) METHOD FOR INHIBITING METASTASIS OF TRIPLE-NEGATIVE BREAST CANCER USING ROSOXACIN

(71) Applicant: National Yang Ming Chiao Tung University, Hsinchu City (TW)

(72) Inventors: Jinn-Moon YANG, Hsinchu City (TW); Chia-Hwa LEE, Taipei City (TW); Jung-Yu LEE, Hsinchu City (TW); Yun-Ti CHEN, Tainan City (TW)

(21) Appl. No.: 18/329,895

(22) Filed: Jun. 6, 2023

(30) Foreign Application Priority Data
Dec. 23, 2022 (TW) 111149673

(51) Int. Cl. A61K 31/4709; A61P 35/04

(52) U.S. Cl. CPC A61K 31/4709; A61P 35/04

(57) ABSTRACT

A method for inhibiting the metastasis of triple-negative breast cancer, which includes administering to a subject in need thereof a pharmaceutical composition containing rosoxacin or a pharmaceutically acceptable salt thereof.

### CROSS-REFERENCE TO RELATED APPLICATION

This application claims priority of Taiwanese Invention Patent Application No. 111149673, filed on Dec. 23, 2022.

### FIELD

The present disclosure relates to a method for inhibiting the metastasis of triple-negative breast cancer using rosoxacin.

### BACKGROUND

Triple-negative breast cancer (TNBC) is a highly aggressive malignancy and is able to spread to distant sites, specifically to the lung, liver, and brain. Advanced triple-negative breast cancer is also known as metastatic triple-negative breast cancer. Triple-negative breast cancer can be treated with surgery in combination with chemotherapy and radiotherapy before metastasis occurs, and has a high survival rate after surgery. However, if metastases occur, there are no effective treatment regimens for triple-negative breast cancer and the survival rates are drastically reduced. Currently, no drugs have been proven to be effective in inhibiting the metastasis of triple-negative breast cancer.

1-ethyl-4-oxo-7-(4-pyridyl)-1,4-dihydroquinoline-3-carboxylic acid (rosoxacin) is a non-fluorinated quinolone antibiotic. Rosoxacin has been approved for treatment of bacterial infections in the respiratory tract, urinary tract, gastrointestinal tract, and central nervous system, and can act effectively against penicillin-resistant strains.

In spite of the aforesaid, there is still a need to develop an effective way for inhibiting the metastasis of triple-negative breast cancer.

### SUMMARY

Accordingly, an object of the present disclosure is to provide a method for inhibiting the metastasis of triple-negative breast cancer, which can alleviate at least one of the drawbacks of the prior art, and which includes administering to a subject in need thereof a pharmaceutical composition containing rosoxacin or a pharmaceutically acceptable salt thereof.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the cell invasion percentages determined in the control groups H1 to H2, experimental group H, and comparative groups H1 to H5 of Example 1, infra, in which the symbol “***” represents p<0.005 (compared with the control group H1), and the symbol “###” represents p<0.005 (compared with the control group H2).

FIG. 2 shows the cell invasion percentages determined in the control groups M1 to M2 and experimental group M of Example 1, infra, in which the symbol “**” represents p<0.01 (compared with the control group M1), and the symbol “##” represents p<0.01 (compared with the control group M2).

FIG. 3 shows the numbers of tumor spheres determined in the control groups H1 to H2 and experimental group H of Example 2, infra, in which the symbol “*” represents p<0.05 (compared with the control group H1), and the symbol “##” represents p<0.01 (compared with the control group H2).

FIG. 4 shows the numbers of tumor spheres determined in the control groups M1 to M2 and experimental group M of Example 2, infra, in which the symbol “#” represents p<0.05 (compared with the control group M2).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for inhibiting the metastasis of triple-negative breast cancer, which includes administering to a subject in need thereof a pharmaceutical composition containing rosoxacin or a pharmaceutically acceptable salt thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats.

In certain embodiments, the triple-negative breast cancer is metastatic triple-negative breast cancer.

In certain embodiments, the triple-negative breast cancer is PD-L1-positive or PD-L1-negative triple-negative breast cancer. In an exemplary embodiment, the triple-negative breast cancer is PD-L1-positive triple-negative breast cancer.

As used herein, the term “metastasis” refers to the growth of a cancerous tumor in an organ or body part, which is not directly connected to the organ of the original cancerous tumor. Triple-negative breast cancer cells mainly have the ability for lung metastasis, brain metastasis, and liver metastasis.

As used herein, the term “pharmaceutically acceptable salt” refers to any salt, which, upon administration to the subject is capable of providing (directly or indirectly) a compound as described herein (i.e., rosoxacin) without undue toxicity, irritation, allergic response and the like. In particular, “pharmaceutically acceptable salt” may encompass those approved by a regulatory agency of the federal or a state government or listed in the U.S. Pharmacopeia or other generally recognized pharmacopeia for use in animals, and more particularly in humans. The preparation of salts can be carried out by methods known in the art.

For instance, the pharmaceutically acceptable salts of rosoxacin may be acid addition salts, base addition salts or metallic salts, and they can be synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture thereof. Examples of the acid addition salts may include mineral acid addition salts such as, for example, hydrochloride, hydrobromide, hydroiodide, hemisulfate, nitrate, and phosphate; and organic acid addition salts such as, for example, acetate, maleate, fumarate, citrate, oxalate, succinate, tartrate, malate, mandelate, methanesulphonate, p-toluenesulphonate, 2-naphtalenesulphonate, and 1,2-ethanedisulphonate. Examples of the alkali addition salts may include inorganic salts such as, for example, ammonium; and organic alkali salts such as, for example, ethylenediamine, ethanolamine, N,N-dialkylenethanolamine, triethanolamine, choline, glucamine, and basic amino acids salts. Examples of the metallic salts may include, for example, sodium, potassium, calcium, magnesium, aluminium, and lithium salts.

According to the present disclosure, the pharmaceutical composition may be formulated into a dosage form suitable for parenteral administration or oral administration using technology well known to those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

The dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials

### 1. Source and Cultivation of Cell Lines

Human breast cancer cell line MDA-MB-231 (ATCC HTB-26) was purchased from American Type Culture Collection (ATCC, Manassas, Va., USA).

In addition, the highly metastatic breast cancer cell line (abbreviated as HMB cells) used in the following experiments was obtained according to the following preparation processes: the MDA-MB-231 cells (2.5×10⁶ cells) were injected into the mammary fat pad of a severe combined immune deficient (SCID) mice (purchased from BioLasco Taiwan Co., Ltd; 4 weeks old, with a body weight of approximately 20 g), and the triple-negative breast cancer cells metastasized to the lung of the SCID mice were harvested after 12 weeks. Next, the triple-negative breast cancer cells (2.5×10⁶ cells) thus obtained were injected into the mammary fat pad of another SCID mice, and the HMB cells metastasized to the lung of the another SCID mice were harvested after 8 weeks.

The MDA-MB-231 cells and HMB cells were respectively grown in Dulbecco's Modified Eagle's Medium (DMEM) (Gibco) supplemented with 10% fetal bovine serum (FBS) (Gibco), and were cultivated in an incubator with culture conditions set at 37° C. and 5% CO₂. Medium change was performed every two to three days. Cell passage was performed when the cultured cells reached 80% to 90% of confluence.

### 2. Quinolone Antibiotics

The quinolone antibiotics used in the following experiments are listed in Table 1.

TABLE 1

| Quinolone antibiotic | Source |
|---|---|
| Rosoxacin | Accela ChemBio Co., Ltd. |
| Norfloxacin | Toronto Research Chemicals |
| Ofloxacin | Sigma |
| Gatifloxacin | Toronto Research Chemicals |
| Clinafloxacin | TargetMol |
| Garenoxacin | Carbosynth |

### General Procedures

### 1. Statistical Analysis

All the experiments described below were performed in triplicates. The experimental data of all the test groups are expressed as mean±standard error of the mean (SEM), and were analyzed using Student's t-test, so as to evaluate the differences between the groups. Statistical significance is indicated by p<0.05.

### Example 1. Evaluation for the Effect of Rosoxacin in Inhibiting Triple-Negative Breast Cancer Cell Invasion

### Methods

The HMB cells prepared in section 1 of “General Experimental Materials” were divided into 8 groups, namely, two control groups (i.e., control groups H1 and H2), one experimental group (i.e., experimental group H), and five comparative groups (i.e., comparative groups H1 to H5). In addition, the MDA-MB-231 cells prepared in section 1 of “General Experimental Materials” were divided into three groups, namely, two control groups (i.e., control groups M1 and M2) and one experimental group (i.e., experimental group M).

Each group of the cells was seeded at a concentration of 1×10⁵ cells per well into respective permeable Transwell® insert (Corning Inc.) for a 24-well plate. Each of the Transwell® inserts had a polycarbonate membrane (pore size: 0.8 μm) pre-coated with 1 mg/ml Matrigel® basement membrane matrix (Thermo Fisher Scientific Inc.). Next, the cells of each group were added with a suitable amount of the treating agent as shown in Table 2 below.

TABLE 2

| | Treating agent | |
|---|---|---|
| | Lipopolysaccharide | Quinolone antibiotic |
| Group | (LPS)(1 μM) | (1 μM) |
| Control group H1 | − | − |
| Control group H2 | + | − |
| Experimental group H | + | Rosoxacin |
| Comparative group H1 | + | Norfloxacin |
| Comparative group H2 | + | Ofloxacin |
| Comparative group H3 | + | Gatifloxacin |
| Comparative group H4 | + | Clinafloxacin |
| Comparative group H5 | + | Garenoxacin |
| Control group M1 | − | − |
| Control group M2 | + | − |
| Experimental group M | + | Rosoxacin |

Thereafter, the Transwell® inserts were placed into the 24-well plate that contained 0.6 mL of DMEM supplemented with 10% FBS in each well, followed by cultivation in a humidified incubator (37° C., 5% CO₂, 95% relative humidity) for 24 hours.

Subsequently, the culture medium in each Transwell® insert was removed, and the respective Transwell® insert was washed with phosphate-buffered saline (PBS). The cells migrating through the polycarbonate membrane and attaching to the bottom side of the polycarbonate membrane were subjected to a fixation treatment with a 4% formaldehyde solution (in PBS) at room temperature for 15 minutes. Then, the non-migrating cells were removed using a cotton swab, followed by conducting staining using a 1% crystal violet solution according to techniques well-known to those skilled in the art. The number of the stained cells was counted under a Leica DMI3000 B inverted microscope (Leica Microsystems, Wetzlar, Germany) at 100× magnification, followed by analysis using Image-Pro Software (version 6.0).

The cell invasion percentage (%) of each group was calculated by substituting the thus obtained number of stained cells into the following Equation (I):

$$A = ( B / C ) \times 10 \; 0 . \qquad ( I )$$

where A=cell invasion percentage (%)
   B=number of stained cells in respective group
   C=number of stained cells in the control group H1 or the control group M1

The data thus obtained were analyzed according to the method described in section 1 of “General Procedures”.

### Results

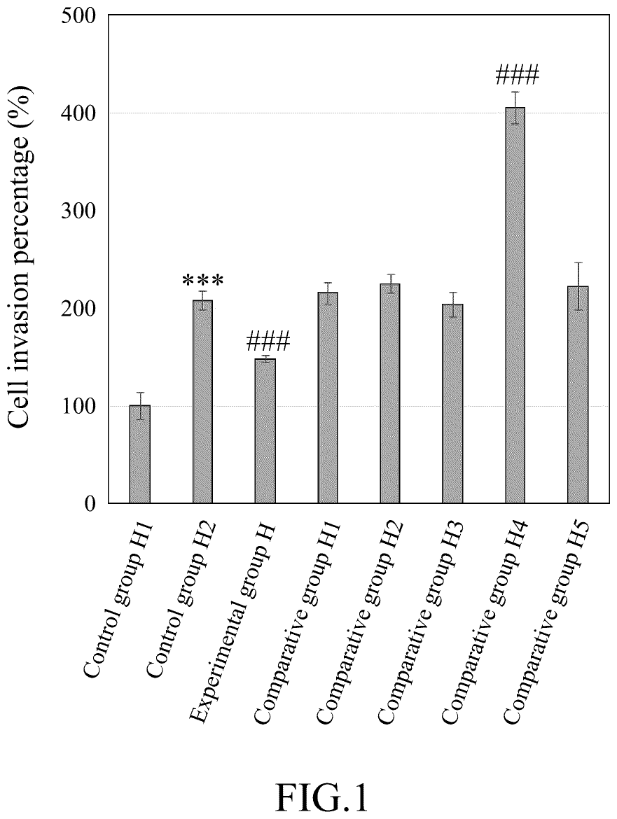

Referring to FIG. 1, the cell invasion percentage determined in the control group H2 was significantly higher than that determined in the control group H1, indicating that HMB cell migration was successfully induced by LPS. In particular, the cell invasion percentage determined in each of the comparative groups H1, H2, H3, and H5 was substantially similar to that determined in the control group H2, and the cell invasion percentage determined in the comparative group H4 was significantly higher than that determined in the control group H2. In addition, the cell invasion percentage determined in the experimental group H was significantly lower than that determined in the control group H2, indicating that rosoxacin can effectively inhibit the migration of HMB cells.

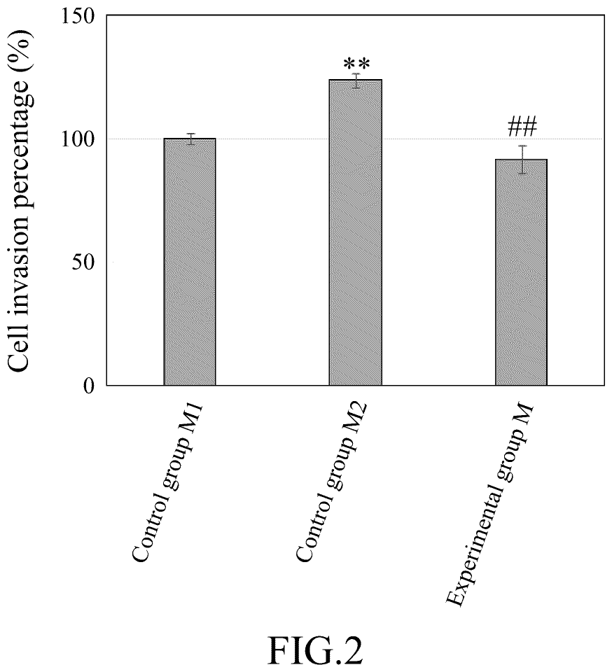

Furthermore, referring to FIG. 2, the cell invasion percentage determined in the control group M2 was significantly higher than that determined in the control group M1, indicating that the migration of MDA-MB-231 cells was successfully induced by LPS. The cell invasion percentage determined in the experimental group M was significantly lower than that determined in the control group M2, indicating that rosoxacin can effectively inhibit the migration of MDA-MB-231 cells.

These results indicate that rosoxacin can exhibit satisfactory efficacy in inhibiting the migration of breast cancer cells (in particular highly metastatic breast cancer cells), and hence is capable of inhibiting the metastasis of triple-negative breast cancer.

### Example 2. Evaluation for the Effect of Rosoxacin in Inhibiting Tumor-Sphere Formation in Triple-Negative Breast Cancer Cells

### Materials

The sphere formation medium used in this example was prepared by supplementing Gibco DMEM/F-12 medium with the ingredients shown in Table 3.

TABLE 3

| Ingredients | Concentration | | Source |
|---|---|---|---|
| B-27 supplement (50X) | 2 | vol % | Invitrogen |
| Human epidermal growth factor | 20 | ng/mL | |
| (hEGF) | | | |
| Basic fibroblast growth factor | 20 | ng/mL | |
| (bFGF) | | | |
| Penicillin | 5 | μg/mL | |
| Streptomycin | 5 | μg/mL | |

### Methods

The HMB cells prepared in section 1 of “General Experimental Materials” were divided into three groups, namely, two control groups (i.e., control groups H1 and H2) and one experimental group (i.e., experimental group H). In addition, the MDA-MB-231 cells prepared in section 1 of “General Experimental Materials” were divided into three groups, namely, two control groups (i.e., control groups M1 and M2) and one experimental group (i.e., experimental group M).

Each group of the cells was incubated in a respective well of a low cell attachment plate (Thermo Fisher Scientific Inc.) containing 2 mL of the sphere formation medium at 2×10⁴ cells/well. Next, the cells of each group were added with a suitable amount of the treating agent as shown in Table 4 below, followed by cultivation in a humidified incubator (37° C., 5% CO₂, 95% relative humidity) for 21 days, so as to induce the formation of tumor spheres.

TABLE 4

Treating agent

| | LPS | Rosoxacin |
|---|---|---|
| Group | (1 μM) | (1 μM) |
| Control group H1 | − | − |
| Control group H2 | + | − |
| Experimental group H | + | + |
| Control group M1 | − | − |
| Control group M2 | + | − |
| Experimental group M | + | + |

The number of tumor spheres was counted under a Leica DMI3000 B inverted microscope (Leica Microsystems, Wetzlar, Germany) at 100× magnification.

The data thus obtained were analyzed according to the method described in section 1 of “General Procedures”.

### Results

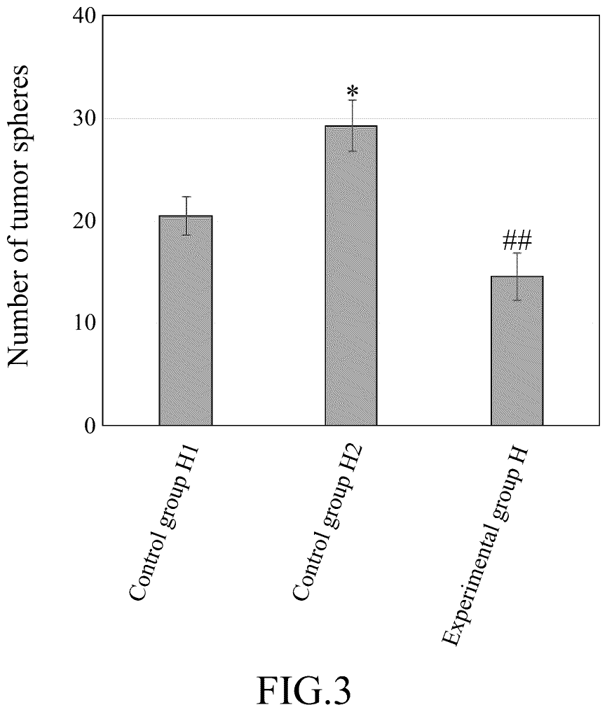

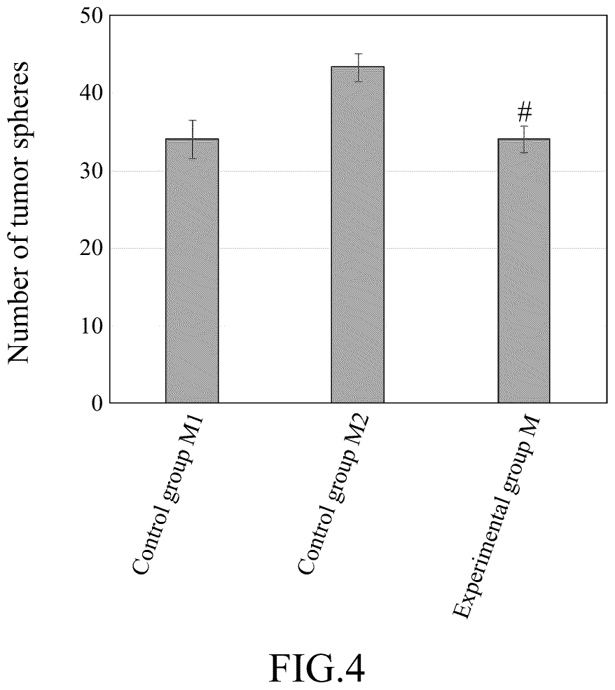

Referring to FIG. 3, the number of tumor spheres determined in the control group H2 was significantly higher than that determined in the control group H1, indicating that the HMB cells were induced to form tumor spheres by LPS. In addition, the number of tumor spheres determined in the experimental group H was significantly lower than that determined in the control group H2, indicating that rosoxacin is capable of inhibiting tumor-sphere formation induced by LPS. Besides, similar satisfactory result was observed with respect to the experimental group M (see FIG. 4).

Summarizing the above test results, it is clear that rosoxacin is capable of effectively inhibiting the migration of triple-negative breast cancer cells (in particular highly metastatic triple-negative breast cancer cells) and inhibiting tumor-sphere formation in triple-negative breast cancer cells, and hence can inhibit the metastasis of triple-negative breast cancer.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for inhibiting the metastasis of triple-negative breast cancer, comprising administering to a subject in need thereof a pharmaceutical composition containing rosoxacin or a pharmaceutically acceptable salt thereof.

2. The method as claimed in claim 1, wherein the pharmaceutical composition is in a dosage form selected from the group consisting of an oral dosage form and a parenteral dosage form.
